(19) United States
(12) Patent Application Publication
(10) Pub. No.: US 2020/0345728 A1
(43) Pub. Date: Nov. 5, 2020

(54) COMPOSITIONS AND METHODS FOR TREATMENT OF ATHEROSCLEROSIS

(71) Applicants: NovMetaPharma Co., Ltd., Seoul (KR); The United States Government as Represented by the Department of Veterans Affairs, Washington, DC (US)

(72) Inventors: Moon Ki Song, Northridge, CA (US); Mysore S. Veena, Los Angeles, CA (US); David Scott Bischoff, Canyon Country, CA (US); Dean Takao Yamaguchi, Woodland Hills, CA (US)

(73) Assignees: NovMetaPharma Co., Ltd., Seoul (KR); The United States Government as Represented by the Department of Veterans Affairs, Washington, DC (US)

(21) Appl. No.: 16/398,421

(22) Filed: Apr. 30, 2019

(51) Int. Cl. A61K 31/4985; A61K 33/30; A61P 9/10; A61K 9/48; A61K 9/20

(52) U.S. Cl. CPC A61K 31/4985; A61K 33/30; A61K 9/20; A61K 9/48; A61P 9/10

(57) ABSTRACT

The present invention relates to a composition for preventing or treating atherosclerosis in a subject, comprising a zinc salt and cyclo-Hispro and a method of preventing or treating atherosclerosis using thereof.

### FIELD OF THE INVENTION

The present invention relates to a composition for preventing or treating atherosclerosis in a subject, comprising a zinc salt and cyclo-Hispro and a method of preventing or treating atherosclerosis using thereof.

### BACKGROUND

Melucci et al. reported consumption of Asphodeline lutea extracts conferred anti-oxidant properties that could play a positive and safe role in human health maintenance (Melucci D, Locatelli, M, Locatelli C, et al., A comparative assessment of biological effects and chemical profile of Italian Asphodeline lutea extracts. Molecules 461: 1-14, 2018). Furthermore, Ho et al. reported that oxidative stress is a common mediator in the pathology of established cardiovascular risk factors (Ho E, Galougahi, K K, Liu C-C, Bhindi R. Biological markers of oxidative stress: Application to cardiovascular research and practice. Redox Biology 1:483-491. 2013). Hemoxygenases-1 (HO-1) can be induced by many stimuli, including heat shock, hyperoxia, and oxidative stress; and represents a powerful endogenous protective mechanism against free radicals, a subgroup of reactive oxygen species (ROS), in a variety of pathological conditions. ROS are formed as a natural byproduct of normal metabolism of oxygen and have important roles in cell signaling and homeostasis. However, during times of cellular stress, including UV or heat exposure, levels of ROS dramatically increases resulting in significant damage to cell structures. During normal human activity—energy production, detoxification of pollutants, and immunologic defense mechanisms, free radicals are produced. These free radicals are unstable molecules that can extract an electron from a neighboring molecule, causing damage in the process. Unchecked free radical production accelerates the pathogenesis of human disease and aging. These free radicals are counter-balanced by antioxidants present in our foods (and supplements) and by induction of anti-oxidant proteins in the body. Dietary antioxidants (such as proanthocyanins found in blueberries and bioflavonoids found in citrus fruits), as well as the induction of endogenous anti-oxidant enzymes, superoxide dismutase and glutathione peroxidase, provide critical protection against free radical damage. Oxidative stress results when this delicate pro-oxidant/anti-oxidant equilibrium is disrupted in favor of the pro-oxidant (free radical) state.

According to a report of Genova Diagnostic (Oxidative Analysis 2.0. Ashville, N.C. 28801-1074, Web: www.GDX.net), accumulating oxidative stress is involved in many pathological processes, including Rheumatoid arthritis, Arthritis, Asthma, Diabetes mellitus, Cancer, Atherosclerosis, Macular degeneration, Chronic Fatigue Syndrome, Inflammatory Bowel Disease, Environmental sensitivity, and Neurodegenerative diseases such as Parkinson's and Alzheimer's disease. Oxygen radicals can induce apoptosis in human polymorphonuclear cells. Apoptosis is also known to regulate ectopic calcification both in vitro and in vivo. The mechanism involved in initiation of calcification is not clearly understood, but is thought to be associated with apoptotic cell death. Vascular calcification is a highly organized process and a prominent feature of atherosclerosis; and when present, is associated with major adverse cardiovascular events with an increased risk of myocardial infarction. Vascular smooth muscle cells (VSMC) apoptosis has been implicated in plaque rupture, coagulation, vessel remodeling, medial atrophy, aneurism formation, and calcification. VSMC calcification, which is also associated with aging, diabetes, uremia, and high serum calcium and phosphate levels, is not a passive process but involves actively reprograming VSMCs by local environment cues into a dynamic range of phenotypes, where the primary drivers are inflammation, oxidative stress, apoptosis, and medial calcification. Bone and cartilage tissues as well as osteoblast- and chondrocyte-like cells are actively present during calcification. As in bone formation, apoptosis and matrix vesicles play an important role in the initiation of vascular calcification. Several mechanisms have been identified including loss of inhibition, induction of bone formation, circulating nucleation complexes, and cell death (Giachelli C M. Vascular calcification mechanisms. J Am Soc Nephrol 15:2959-2964, 2004); and this author has also suggested that vascular calcification can be reversible which can be developed in the future studies. Yagishita et al. reported that increased nuclear factor-erythroid 2-related factor (Nrf2) signaling suppresses hypothalamic oxidative stress and improves insulin and leptin resistance in severely insulin resistant Trsp-KO mice (Yagishita Y, Uruno A, Fukutomi T. et al. Nrf2 improves leptin and insulin resistance provoked by hypothalamic oxidative stress. Cell Reports 18:2030-2044, 2017). Thus, Nrf2 harbors the potential to prevent the onset of diabetes by reducing hypothalamic oxidative stress; and in fact, decreased oxidative stress supports Diabetes mellitus treatment. Insulin signaling is an important factor for the prevention of apoptosis in smooth muscle cells and decreased insulin sensitivity in the artery, due to diabetes, may increase smooth muscle cell death and cause unstable plaque formation associated with cardiovascular disease. It is well known that most diabetic patients end up dying from complications of cardiovascular disease and atherosclerosis. Oxygen radicals induce apoptosis in VSMCs, and smooth muscle cell death may account for medial calcification found in arteries, frequently seen in diabetics and during end stage renal failure, and which is positively correlated with the prognosis of these diseases. Insulin signaling in the arteries is diminished in hyper-insulinemic obese animals with insulin resistance; and therefore, VSMCs may also by susceptible to apoptosis in insulin-resistant type 2 diabetes. Thus, induction of anti-oxidant mechanisms in diabetics may also reduce cardiovascular disease and atherosclerosis incidence.

Most methods for treating atherosclerosis involve uses of prescription medication. For instance, the group of medications known as statins are prescribed for treating atherosclerosis in patients with high cholesterol but its effects in women and people over the age of 70 are unclear. Niacin, a vitamin, has also been prescribed for treating atherosclerosis but it causes flushing of the skin and increases blood sugar levels which can be risky for diabetic patients. Drugs for limiting the absorption of cholesterol like Ezetimibe have also been prescribed for patients with atherosclerosis but its efficacy in reducing the risk of heart attacks and strokes in those patients is unclear.

Other methods for treating atherosclerosis include medical procedures such as surgical stenting, surgical excision of the plaque, ablation of the plaque, and bypass surgery/grafting. These procedures are costly and not without risks and limitations. Stenting of the arterial wall comes with the risks of blood clots and the stent itself can become blocked over time. Surgical excision and ablation can release plaque particles that can lead to obstruction of arteries leading to the brain causing a stroke. Grafting the arteries with autologous blood vessels can also lead to other complications such as stroke, heart attacks, reduced kidney function and irregular heartbeats. All these surgical procedures are also severely limited by targeting only specific arteries and leaving other arteries that may be affected by atherosclerosis untreated.

Atherosclerosis can be prevented or mitigated by modification of risk factors such as smoking cessation, increase exercise, managing weight in obese patients, lowering blood pressure, monitoring blood lipid levels and changing poor dietary habits. Additionally, patients at risk of developing atherosclerosis are advised to reduce their cholesterol and saturated fat intake by substituting their diets with unsaturated fatty acids found in natural oils such as olive oil. Olive oil and other naturally occurring oils, however, also contain other undesirable fatty acids that has been known to contribute to atherosclerosis.

Despite the advances in the study, prevention, and treatment of atherosclerosis, it remains a leading cause of death or disability in people. Accordingly, there exists a need for effective treatment of atherosclerotic plaques without the need for invasive medical procedures and risky side effects from prescription pharmaceuticals.

### BRIEF DESCRIPTION OF THE INVENTION

It has been found that a composition including a zinc salt and cyclo-Hispro (i.e. Cyclo-Z) can be used to effectively treat atherosclerosis. Cyclo-Z initially induces reactive oxygen species (ROS) generation leading to nuclear factor-erythroid 2-related factor (Nrf2) dependent induction of the antioxidant genes HO-1. The present inventors also found that antioxidant activity induced Cell viability by preventing apoptosis, which leads to anti-calcification of Vascular Smooth muscle Cells (VSMC) apoptosis that induces development of calcified plagues. These inhibitory activities of VSM calcification is prevention of prominent feature of atherosclerosis.

In one aspect, the present invention provides a method of preventing or treating atherosclerosis in a subject, the method comprising administering a therapeutically effective amount of a composition including a zinc salt and cyclo-Hispro to the subject.

In another aspect, the present invention provides a composition for preventing or treating atherosclerosis, comprising a zinc salt and cyclo-Hispro.

In still another aspect, the present invention provides a method of inhibiting inflammatory M₁ macrophage and/or inducing anti-inflammatory M₂ macrophage activation, comprising administering a therapeutically effective amount of a composition including a zinc salt and cyclo-Hispro.

In yet another aspect, the present invention provides a composition for inhibiting inflammatory M₁ macrophage and/or inducing anti-inflammatory M₂ macrophage activation, comprising a zinc salt and cyclo-Hispro.

In still another aspect, the present invention provides a method for preventing or treating of other metabolic diseases, neurodegenerative diseases or obesity in a subject, comprising administering a therapeutically effective amount of a composition including a zinc salt and cyclo-Hispro to the subject.

In yet another aspect, the present invention provides a composition for preventing or treating of other metabolic diseases, neurodegenerative diseases, or obesity in a subject, comprising a zinc salt and cyclo-Hispro.

According to a preferred embodiment of the present invention, the zinc salt may comprise a zinc cation and an anion.

According to another preferred embodiment of the present invention, the weight ratio of the zinc salt to cyclo-Hispro may be 1:0.1 to 10.

According to still another preferred embodiment of the present invention, the composition is in the form of a tablet or capsule.

According to yet another preferred embodiment of the present invention, each tablet or capsule may contain from 2 mg to 100 mg of cyclo-Hispro.

According to yet another preferred embodiment of the present invention, each tablet or capsule may contain from 10 mg to 100 mg of zinc.

According to yet another preferred embodiment of the present invention, the composition may induce ROS formation from hydrogen peroxide (H₂O₂).

According to yet another preferred embodiment of the present invention, the composition may induce factor-erythroid 2-related factor (Nrf2).

According to yet another preferred embodiment of the present invention, the composition may induce antioxygen hemoxygenase-1 (HO-1).

According to yet another preferred embodiment of the present invention, the composition may prevent apoptosis or cell death.

According to yet another preferred embodiment of the present invention, the composition may prevent vascular smooth muscle cells (VSMC) calcification.

According to yet another preferred embodiment of the present invention, the other metabolic diseases may be selected from the group consisting of rheumatoid arthritis, asthma, diabetes mellitus, cancer, macular degeneration, immune system diseases, inflammation, osteoporosis, and inflammatory bowel diseases, and the neurodegenerative disease including Alzheimer's disease.

### BRIEF DESCRIPTION OF THE FIGURES

FIG. 1 shows that direct anti-oxidant activities of CHP (DPPH: 2,2-diphenyl-1-picrylhydrazyl; DMSO: Dimethyl sulfoxide (Control Solvent); *P<0.05; **P<0.01; ***P<0.001 compared to DPPH treated solution).

FIG. 2 shows heme oxygenase-1 expression in vascular smooth muscle cells (BGP: β-glycerol phosphate (10 mM); M1CM: Conditional medium (1:1 diluted CM with fresh medium); **P<0.01; ***P<0.001 compared to control untreated).

FIG. 3 shows Nrf2 mRNA expression in vascular smooth muscle cells (**P<0.01 compared to control untreated).

FIG. 4 shows anti-inflammatory interleukin-10 (IL-10) expression in vascular smooth muscle cells (**P<0.01 compared to control untreated).

FIG. 5 shows TNFα expression in vascular smooth muscle cells (**P<0.01 compared to control untreated).

FIG. 6 shows effects of inflammatory (M₁) or anti-inflammatory (M₂) activation on levels of TNFα mRNA expression in the RAW264.7 macrophage cell line (IFN: 20 ng/mL Interferon-γ; LPS: 100 ng/mL lipopolysaccharide; IL-4: 20 ng/mL Interleukin-4; Cyclo-Z: 50 μM CHP and 10 μM ZnCl₂; ***P<0.001 (estimated assuming correct mean value)).

FIG. 7 shows effects of inflammatory (M₁) or anti-inflammatory (M₂) activation on levels of CD11c mRNA expression in the RAW264.7 macrophage cell line (***P<0.001 untreated control (estimated assuming correct mean value)l; ### P<0.001 compared to IL-4 treatment (estimated assuming correct mean value)).

FIG. 8 shows effects of inflammatory M₁) or anti-inflammatory (M₂) activation on levels of CD206 mRNA expression in the RAW264.7 macrophage cell line (### P<0.001 compared to control untreated (estimated assuming correct mean value); ***P<0.001 compared to IL-4 treatment (estimated assuming correct mean value)).

FIG. 9 shows effect of Cyclo-Z on VSMCs Cell viability in serum-free cultures at 7 days (Cyclo-Z: 100 μM CHP and 10 μM Zinc).

FIG. 10 shows effect of Cyclo-Z (50 uM CHP/10 uM zinc) on B-cell activation in mouse spleen tissues.

FIG. 11 shows effects of Cyclo-Z on phagocytic activity of inflammatory (M₁) or anti-inflammatory (M₂) activated RAW264.7 macrophage cells.

FIG. 12 shows effects of Cyclo-Z or Zinc alone on phagocytic activity of inflammatory (M₁) activated RAW264.7 macrophage cells.

FIG. 13 shows VSMC calcification induced with 2.5 mM CaCl₂, 10 mM BGP in VSMC cultures directly treated with Cyclo-Z (6 weeks) (11.7 μg/mL CHP=50 μM CHP; 117 μg/mL CHP=0.5 mM CHP; 1.4 μg/mL Zinc=10 μM Zinc).

FIG. 14 shows calcification of VSMCs dependent on inflammatory factors present in conditioned medium from M₁ (inflammatory) or M₂ (anti-inflammatory) RAW264.7 cell cultures with or without Cyclo-Z added during macrophage activation.

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

It is noted that, as used in this specification and the appended claims, the singular forms “a,” “an,” and “the,” include plural referents unless expressly and unequivocally limited to one referent. As used herein, the term “include” and its grammatical variants are intended to be non-limiting, such that recitation of items in a list does not exclude other like items that can be substituted or other items that can be added to the listed items.

Through the whole document of the present invention, a phrase in the form “A and/or B” means “A or B” or “A and B”.

“Vascular calcification” as used herein, means formation, growth or deposition of extracellular matrix hydroxyapatite (calcium phosphate) crystal deposits in blood vessels. Vascular calcification encompasses coronary, valvular, aortic, and other blood vessel calcification. The term includes atherosclerotic and medial wall calcification.

“Atherosclerotic calcification” means vascular calcification occurring in atheromatous plaques along the intimal layer of arteries.

The term “treatment” or “treating” includes the administration, to a person in need, of an amount of Cyclo-Z, which will inhibit, decrease or reverse development of a pathological vascular calcification condition. “Inhibiting,” in connection with inhibiting vascular calcification, is intended to mean preventing, retarding, or reversing formation, growth or deposition of extracellular matrix hydroxyapatite crystal deposits. Treatment of diseases and disorders herein is intended to also include therapeutic administration of Cyclo-Z of the invention (or a pharmaceutical acceptable salt, derivative thereof) or a pharmaceutical composition containing Cyclo-Z to a subject (i.e., an animal, for example a mammal, such as a human) believed to be in need of preventative treatment, such as, for example, atherosclerosis. Treatment also encompasses administration of Cyclo-Z or the pharmaceutical composition to subjects not having been diagnosed as having a need thereof, i.e., prophylactic administration to the subject. Generally, the subject is initially diagnosed by a licensed physician and/or authorized medical practitioner, and a regimen for prophylactic and/or therapeutic treatment via administration of Cyclo-Z or the compositions of the invention is suggested, recommended or prescribed.

The phrase “therapeutically effective amount” is the amount of Cyclo-Z that will achieve the goal of improvement in disorder severity and the frequency of incidence. The improvement in disorder severity includes the reversal of vascular calcification, as well as slowing down the progression of vascular calcification.

As used herein, the term “subject” is intended to mean a human or other mammal, exhibiting, or at risk of developing, calcification. Such an individual can have, or be at risk of developing, for example, vascular calcification associated with conditions such as atherosclerosis, stenosis or restenosis. The prognostic and clinical indications of these conditions are known in the art.

As described above, there exists a need for effective treatment of atherosclerotic plaques without the need for invasive medical procedures and risky side effects from prescription pharmaceuticals.

The present inventors confirmed that a composition comprising a zinc salt and cyclo-Hispro can be used to effectively treat atherosclerosis and have devised a solution to the aforementioned problems by providing a method for treating atherosclerosis comprising using a composition comprising a zinc salt and cyclo-Hispro.

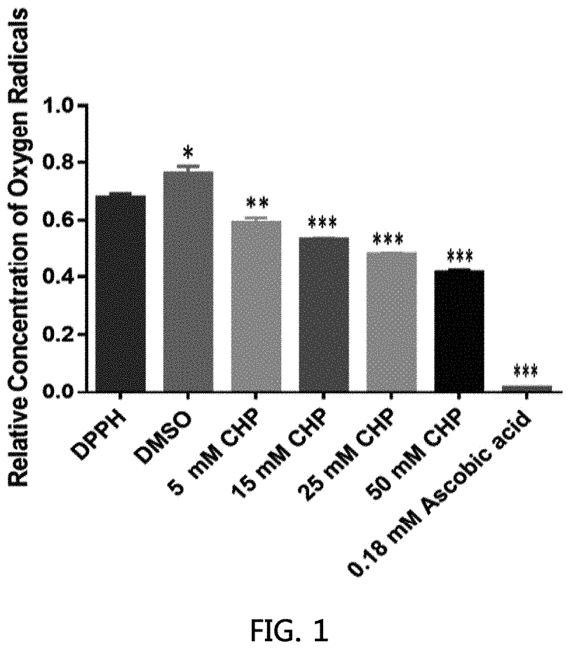

Cyclo-Z treatment induces hydrogen peroxide to be metabolized to hydroxy radical and water and stimulate reactive oxygen species (ROS) production (Table 1). ROS is eventually eliminated through interaction with CHP (in the Cyclo-Z formulation) functioning as a direct anti-oxidant compound binding ROS and metabolized to eliminate ROS in the biological system (FIG. 1). More importantly, in VSMC cultures in which cells were treated with high glucose or hydrogen peroxide to simulate dysregulated glucose metabolism and oxidative stress, Cyclo-Z provide protection to the oxidative stress resulting in increased cell viability and numbers (Table 1).

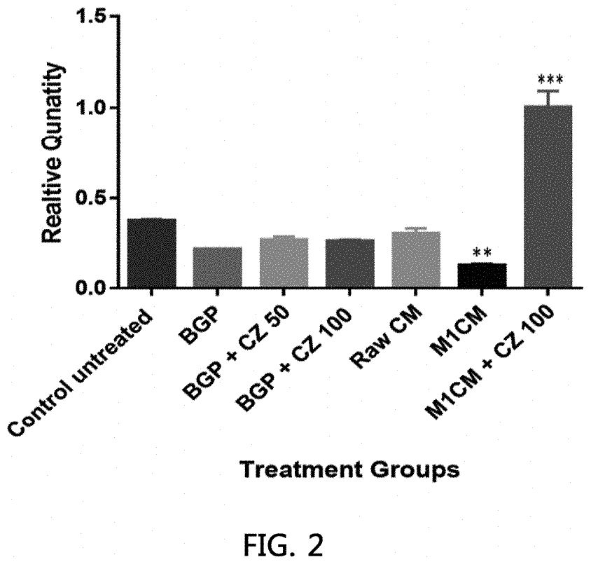

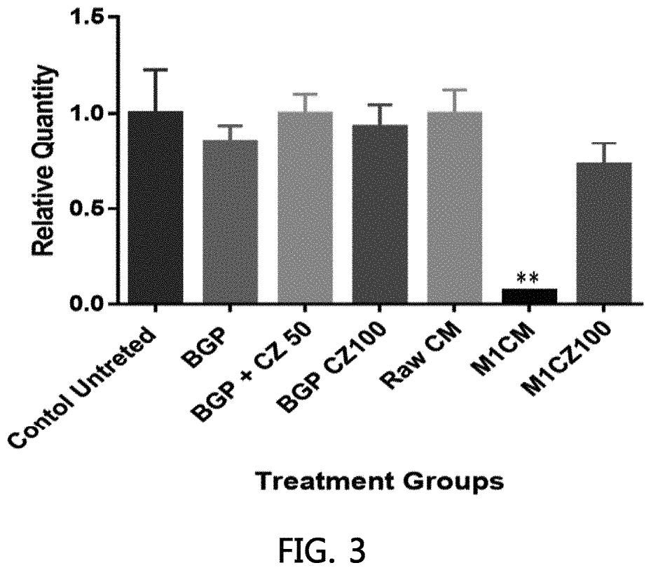

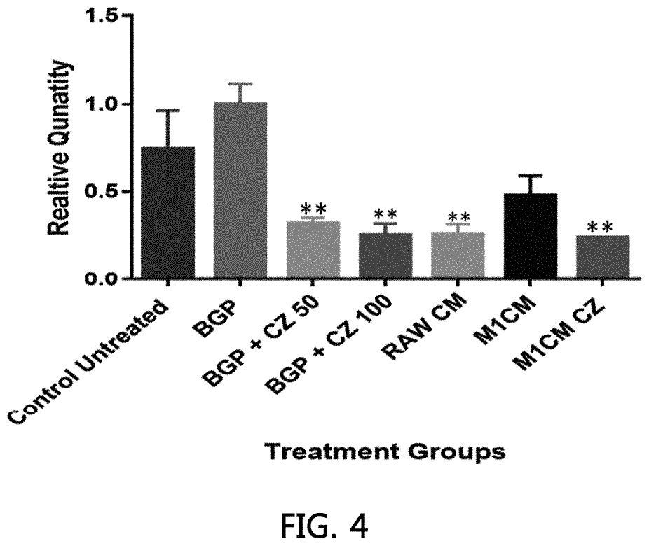

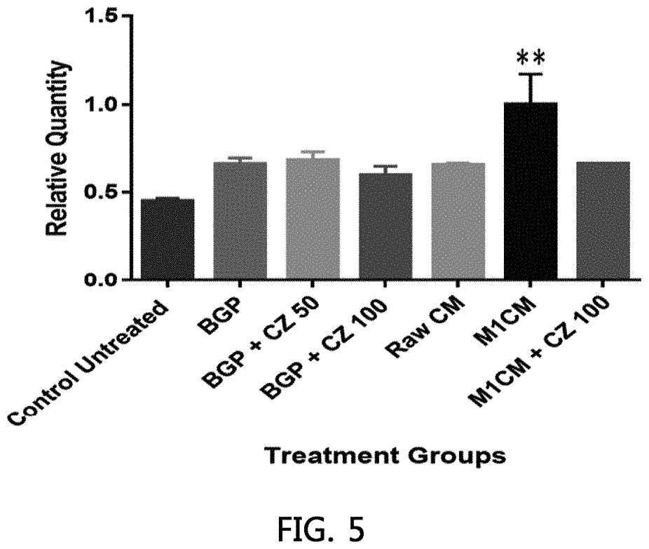

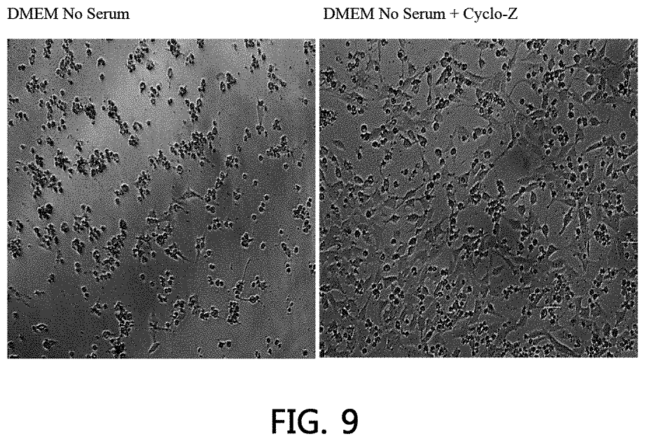

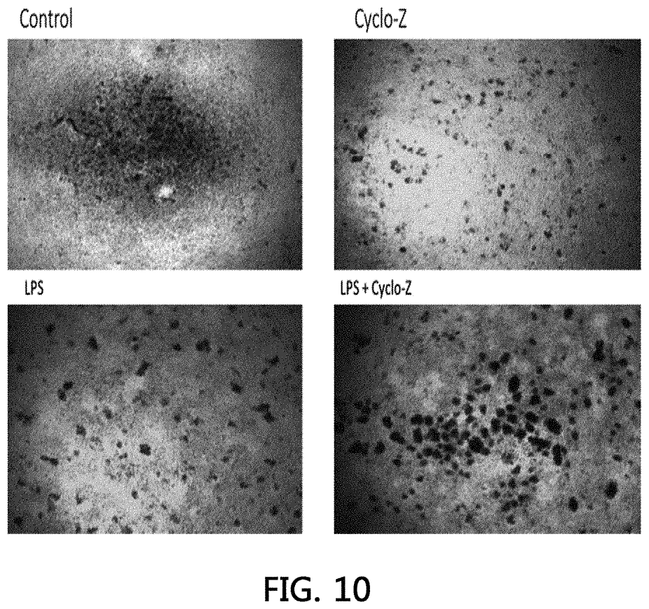

FIGS. 2 to 5 indicate that Cyclo-Z treatment is involved in underlying regulatory mechanism of inflammatory cytokines that can lead to understanding of the interactions between immune responses and metabolic diseases such as, diabetes, obesity, inflammation, atherosclerosis etc. When VSMCs were incubated in conditioned medium (CM) collected from 48-hr RAW264.7 macrophage cell cultures induced to the M₁ inflammatory phenotype for 3 days, Heme Oxygenase (HO) and Nrf2 mRNA levels decreased (FIGS. 2 and 3) but IL-10 and TNFα levels increased with β-glycerophosphate (BGP) treatment (FIGS. 4 and 5). BGP is an organic phosphate donor and has been used in culture media for mesenchymal stem cell differentiation to osteoblast-type cells as a source of phosphate for subsequent mineralization. When Cyclo-Z was added with BGP to VSMC cultures, relative quantity of HO-1 and Nrf2 slightly increased (FIGS. 2 and 3), TNFα levels did not change (FIG. 5), however, IL-10 levels very significantly decreased (FIG. 4) compared to the cells incubated with BGP alone. All four genes HO-1, NRF2, TNFα, and IL-10 are expressed in VSMC cultures. Incremental addition of Cyclo-Z alone (up to 500 mM) did not change the expression levels of these genes (data not shown). However, when conditioned medium from inflammatory M₁ macrophages was added, HO-1 and Nrf2 levels decreased very significantly (FIGS. 2 and 3) but IL-10 and TNFα levels significantly increased (FIGS. 4 and 5). When 50 μM Cyclo-Z was added to these media, HO-1 and Nrf2 levels very significantly increased but IL-10 and TNFα levels significantly decreased (FIGS. 4 and 5). FIGS. 2 to 5 clearly indicated that Cyclo-Z strongly antagonized inflammatory and stimulated anti-oxidant activities of M₁ macrophages by inducing Nrf2 to prevent oxidative stress. Cyclo-Z treatment also significantly protected cell viability (Table 1). It is well established that oxygen radicals induce apoptosis in VSMCs and that smooth muscle cell death may induce medial calcification in arteries of diabetics resulting in death from major adverse cardiovascular events. FIGS. 9 and 10 demonstrated reduced calcification (less red staining) of VSMCs in the Cyclo-Z treatment which may be due to reduced oxidative stress and apoptosis of the VSMCs.

The importance of regulating Nrf2, heme oxygenase-1, and inflammatory cytokines in the control of inflammation and oxidative stress is as followings:

1. Heme oxygenase (HO): HO is an enzyme that catalyzes the degradation of heme, producing biliverdin, ferrous iron, and carbon monoxide. Three isomers of HO are known as HO-1, HO-2, and HO-3. HO-1 is an inducible isoform that is upregulated in response to stresses such as oxidative stress, hypoxia. heavy metals, inflammatory cytokines, etc. HO-2 is a constitutive isoform that is expressed under homeostatic conditions. HO-3 is not catalytically active, but is thought to work in oxygen signaling. HO activity is induced by oxidative stress and the present inventors therefore studied only HO-1. HO-1 has been shown to exhibit important anti-oxidant and anti-inflammatory properties, which results in an anti-atherogenic effect. The remarkable cardioprotective effects of HO-1 are due to its ability to regulate inflammatory processes, cellular signaling, and mitochondrial function ultimately mitigating myocardial tissue injury and the progression of vascular-proliferative disease. HO-1 is associated with low-grade chronic inflammation and stress stimuli; and, therefore, plays an important role in cellular protection by reducing inflammatory, oxidative and endoplasmic reticulum (ER) stress, and by anti-apoptotic effects in metabolic diseases such as insulin resistance, Type 2 diabetes, and obesity. The HO system also abates inflammation through suppression of macrophage-infiltration and abrogation of oxidative/inflammatory transcription factors like NF-KB, INK and active protein-1, and insulin signaling. However, in contrast to the beneficial effects of HO-1 against ectopic calcification, HO-1 also regulates the inflammatory reaction, and increased neuronal cell proliferation and apoptosis in rats after intracerebral hemorrhage (ICH). Inhibition of HO-1 activity with the HO-1 inhibitor, zinc protoporphyrin (ZPP) does have a protective effect on ICH rats which is thought to be due to HO-1 regulation of the inflammatory reaction and neuronal cell apoptosis as HO-1 functions as an anti-inflammatory agent. HO-1 deficiency in normal cells enhances DNA damage and carcinogenesis. Thus, it may also have impacts on cancer progression through modulating tumor microenvironment and inducing M₁ (inflammatory) macrophages to attack the tumors. HO-1 also plays a key role in mediating against oxidant-mediated lung injury. On the other hand, HO-2 deficiency enhances Streptozotocin-induced renal dysfunction and morphologic damage; and upregulation of HO-1 in HO-2 -deficient mice rescued and prevented the morphologic damage. Therefore, the catalytic mechanisms of constitutively expressed HO-2 appear to be similar to that of HO-1. As shown in FIG. 2, Cyclo-Z treatment in a conditioned medium collected from 48 hours old RAW264.7 cell culture with M₁ macrophage phenotype (inflammatory phenotype) (M₁ CM), VSMCs HO-1 mRNA levels increased significantly which may be beneficial for preventing vascular calcification.

2. Reactive oxygen species (ROS): In biological context, ROS are formed as a natural byproduct of the normal metabolism of oxygen and have important roles in cell signaling and homeostasis. Free radical damage to proteins/enzymes in the body often results in lipid peroxidation and loss of enzyme activity. Thus, antioxidant manipulation may be helpful in the prevention or severity of many diseases. The network of antioxidants in the body is complex. Anti-oxidant compounds that serve as electron donors break the chain reaction of free radicals, sacrificing their own electrons to inactive the free radicals without turning into free radicals themselves. ROS have been extensively implicated in T-cell hypo-responsiveness, apoptosis, and activation. Thus, the characterization of the precise mechanisms by which ROS are involved in the regulation of T-cell functions is important for understanding immune response and for the new development of new therapeutic treatments against immune-mediated diseases. However, it now clear that ROS-induced programmed cell death or necrosis, induces or suppress the expression of many other genes, and activates cell signaling cascades, such as those involving mitogen-activated protein kinases. Thus, ROS are not only harmful agents that cause oxidative damage in pathologies, they also have important roles as regulatory agents in a range of biological phenomena. It is known that while prolonged exposure to high ROS concentrations may lead to various disorders, low ROS concentrations exert beneficial effects regulating cell signaling cascades. Metallic nanoparticles lead to different biological functions resulting in differential potentials for the generation of ROS. In the intermembrane space, metallic enzymes such as Cu, Mn and Zn superoxide dismutase (SOD), catalyze the conversion of superoxide anions into H₂O₂. Although reactive oxygen species (ROS) are increased by the treatment with Cyclo-Z for hydrogen peroxide degradation (Table 1), Cyclo-(his-pro) (CHP) can act as a direct anti-oxidant in an in vitro anti-oxidant assay in a CHP concentration-dependent manner (FIG. 1). Thus, Cyclo-Z treatment may be a very important anti-oxidant agent in the treatment of various metabolic diseases thru acting as a potent pro-oxidant which generate reactive oxygen radicals via a transition metal (zinc) catalyzed reaction, or indirectly to modulate redox status by upregulating of anti-oxidant proteins and enzymes.

3. Nuclear factor E2-related factor 2 (Nrf2): Nrf2 activation plays a largely protective role for health benefits protecting against oxidative stress-induced endothelial tissues injuries. In the study of Ruotsalainen et al. (Ruotsalinen A-K, Inkala M, Partanen M E et al. The absence of macrophageNrf2 promotes early atherogenesis. Cardiovascular Res 98:107-115, 2013), macrophage-specific loss of Nrf2 promotes atherogenesis in the early phases of lesion development. Nrf2 has also been shown to be involved in the protection against macrophage foam cell formation which can develop into a myocardial infarction by blocking normal blood flow in the blood vessel. Therefore, Nrf2 can serve as a therapeutic target in the treatment of cardiovascular diseases. However, it has been reported that Nrf2 in some case is pro-atherogenic in mice, despite its anti-oxidative function. The net pro-atherogenic effect of Nrf2 may be mediated via positive regulation of CD36 that is a scavenger receptor responsible for recognition of modified LDL, and therefore, plays an important role in plaque development. This data was supported by Mimura and Itoh (Mimura J and Itoh K. Role of Nrf2 in the pathogenesis of atherosclerosis. Free Radic Biol Med 88 (Pt B): 221-232, 2015) indicating that Nrf2 exhibit both pro-and anti-atherogenic effects in experimental animal models, and that the Nrf2 pathway could be a promising target to prevent atherosclerosis due to its regulation of lipid metabolism, particularly plasma cholesterol levels which reacts with macrophage-mediated foam cells. Thus, working to understand the complex interplay among Nrf2 oxidative stress and adipose biology could lead to a variety of possible treatment for obesity and other related disorders. Interestingly, Cyclo-Z treatment stimulate Nrf2 anti-oxidant activity (Table 1) and also improves obesity control in animal models. VSMC incubated in conditional medium collected from 48 hours old RAW264.7 cells culture with M₁ macrophage phenotype (inflammatory) (M₁CM) had decreased levels of HO-1 (FIG. 2) and Nrf2 mRNA (FIG. 3). Addition of Cyclo-Z during generation of the M₁CM conditioned medium restored VSMC basal levels of Nrf2 expression (FIG. 3), and significantly increased levels of HO-1 (FIG. 2) compared to levels of M₁CM medium alone.

It is also has been reported that low levels of Nrf2 are involved in the development of oxidative stress and redox status disbalance in diabetic patients. Bolstering antioxidant defenses through modulation of Nrf2 represent a new class of therapy for diabetic nephropathy. Tan et al. (Tan Y, Ichikawa T, Li J, et al. Diabetic down regulation of Nrf2 activity via ERK contributes to oxidative stress-induced insulin resistance in cardiac cells in vitro and in vivo. Diabetes 60:625-633, 2011) reported that Extracellular Signal-Related Kinase (ERK)-mediated suppression of Nrf2 activity leads to the oxidative stress-induced insulin resistance in adult cardiomyocytes and down-regulated glucose utilization in the diabetic heart. The Nrf2/Keap1/ARE master antioxidant pathway has been implicated in diabetic damage to the pancreas, heart, and skin among other cell types of tissues. These data show that Nrf2 affects a variety of organs, and since Cyclo-Z treatment improves Nrf2 metabolism shows Cyclo-Z's potential to regulate or improve disease states by modulation of NrF2 (FIG. 3). Thus, Cyclo-Z treatment improves expression of Nrf2 and may be beneficial in treating atherosclerosis thru induction of Nrf2.

4. Interleukin-10 (IL-10): IL-10 is an important molecule with a central role in immunity and regulates the activities of macrophages. Thus, development of agents that can selectively affect a very specific biological action of IL-10 may provide significant benefit in treating autoimmune and inflammatory diseases. IL-10 is made primarily by macrophages and T lymphocytes of the Th2 subtype. Its major functions include inhibition of macrophage activation as-well-as inhibition of pro-inflammatory cytokines and cyclooxygenase-2 expression. Lack of IL-10 leads to impaired axon regeneration and poor recovery of motor and sensory function in sciatic nerve crush injured mice and may also play a role in atherogenesis exerting an protective effect on plaque progression, rupture, and thrombosis by influencing the local inflammatory process. In atherogenesis, macrophage foam cell formation is modulated by pathways involving both uptake and efflux of cholesterol and bone marrow cell-transduced macrophage IL-10 production can inhibit plaque formation. IL-10 deficiency plays a deleterious role in atherosclerosis in the early phase of lesion development when proteolytic and procoagulant activity was elevated in advanced lesion. Foam cell formation is a key event in atherosclerosis, and apoptosis of these lipid-laden cells may promote plaque destabilization. IL-10 expression may contribute to plaque stabilization by stimulating the anti-apoptotic Bfl-l and Mcl-l genes in acute c oronary syndrome patients. In addition, the secretion of IL-10 by both malignant and immune cells promotes the progression of lung tumors. In study of the present invention, when M₁ macrophage conditioned medium was incubated with VSMCs, IL-10 levels increased. However, with addition of Cyclo-Z IL-10 mRNA levels significantly decreased (FIG. 4) suggesting that Cyclo-Z initially induces IL-10 under inflammatory (M1CM) conditions to regulate the inflammatory condition. However, after Cyclo-Z has been added, inflammation was quickly reduced and IL-10 production is no longer needed and therefore levels of IL-10 decrease.

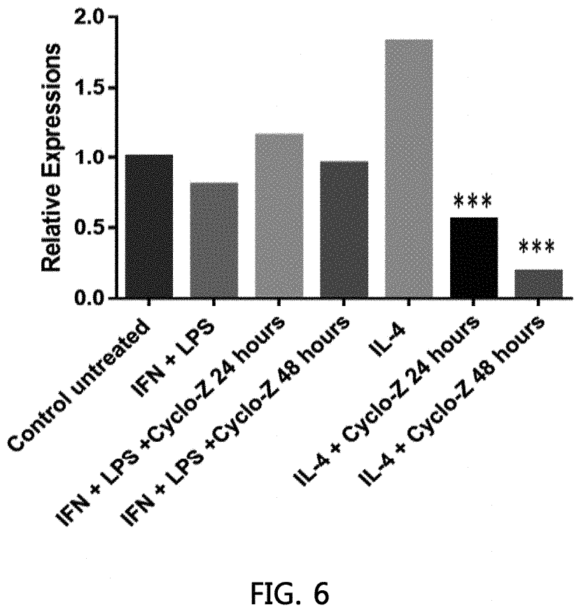

5. Tumor necrosis factor alpha (TNFα): TNFα is a cell signaling protein involved in systematic inflammation and is one of the cytokines that make up the acute phase reaction. It is produced chiefly by activated macrophages; although, it can be produced by many other cell types such as CD⁴⁺ lymphocytes, NK cells, neutrophils, mast cells, eosinophils, and neurons. Modulating TNFα-induced expression of Vascular Cell Adhesion Molecule 1 (VCAM-1), Intracellular Adhesion Molecule-1 (ICAM-1), and fractalkine by inhibition of TNFα activation reduces inflammation in human vascular endothelial cells. The VCAM-1 protein mediates the adhesion of lymphocytes, monocytes, eosinophils, and basophils to vascular endothelium and may also play a role in rheumatoid arthritis. ICAM-1 is an endothelial- and leukocyte-associated transmembrane protein known for its importance in stabilizing cell-cell interactions and facilitating leukocyte endothelial transmigration. More recently, ICAM-1 has been characterized as a site for the cellular entry of human rhinovirus. ICAM-1 ligation produces pro-inflammatory effects such as inflammatory leukocyte recruitment by signaling through cascades involving many kinases, including the kinase p56lyn. TNFα also contributes to metabolic dysregulation by impairing both adipose tissue function and its ability to store excess fuel. Thus, in addition to its role in inducing insulin resistance in adipose tissues, its local actions can impact on whole body insulin sensitivity through increased FFA and altered adipokine production. Cyclo-Z treatment inhibited TNFα production in VSMCs treated with inflammatory M₁ macrophage conditioned medium (FIG. 5). When we determined the effects of the M₁ (inflammatory) and M₂ (anti-inflammatory) cytokines directly on expression of TNFα in the macrophage cells, M₁ treatment (IFN+LPS) did not significantly induce TNFα mRNA; however, M₂ treatment (IL-4) did induce TNFα mRNA. Macrophage expression of TNFα mRNA was inhibited by Cyclo-Z at both 24 and 48 hr under anti-inflammatory M₂ conditions (FIG. 6). These data suggest that TNFα protein is not the compound directly responsible for the M₁CM inflammatory condition in VSMCs, but TNFα is induced by an unknown macrophage inflammatory inducer(s) in the VSMCs. Cyclo-Z can inhibit TNFα expression in macrophage cultures under M₂ conditions (IL-4). Thus, the actions of Cyclo-Z may be specific for the cell type (macrophage or VSMC) and indicates that Cyclo-Z treatment may have different roles under different cell conditions.

6. Effects of Lipopolysaccharide (LPS), Interferon (IFN), Interleukin-4 (IL-4), and Cyclo-Z treatment on CD11c, CD206, IL-10 and TNFα expression: Both LPS and IFNs are inflammation stimuli and are used to induce an M₁ inflammatory phenotype in macrophages. LPS acts as the prototypical endotoxin because it binds the CD4/TLR4/MD₂ receptor complex in many cell types, specifically in monocytes, dendric cells, macrophages, and B cells, which promotes the secretion of pro-inflammatory cytokines, nitric oxide, and eicosanoids. More importantly, LPS has been shown to induce macrophage expression of TNFα, VCAM-1, ICAM-21, and nuclear factor kappa B (NF-κB) signaling associated with inflammation in atherosclerotic mice. Interferons (IFNs) are key inflammatory mediators released by host cells in response to the presence of several pathogens and are also expressed at high levels in atherosclerotic lesions. However, the precise role of IFNγ in atherosclerosis is complex, with both pro- and anti-atherogenic actions being identified reflecting roles in injury, immune response, inflammatory amplification, remodeling, and potential restitution.

Interleukin-4 (IL-4) is a cytokine that regulates multiple biological functions including proliferation, differentiation, and apoptosis in several cell types of haematopoietic and non-haematopoietic origin. It has a critical role in the regulation of Th₀ cell differentiation during a normal immune response, and IL-4-driven Th₂ cells direct host responses against parasitic infections. It has been demonstrated that IL-4 produced by antigen-primed Th₂ cells has a major regulatory influence on the inflammatory response following infection with the antigen-bearing pathogens (McGuirk P, Mills K H G. A regulatory role of Interleukin 4 in differential inflammatory responses in the lung following infection of mice primed with Th1 or Th2 inducing Pertussis vaccines. Infection and Immunity. 68(3):1383-1390, 2000). In these studies (McGuirk P, Mills K H G. A regulatory role of Interleukin 4 in differential inflammatory responses in the lung following infection of mice primed with Th1 or Th2 inducing Pertussis vaccines. Infection and Immunity. 68(3):1383-1390, 2000), knockout mice revealed neutrophil and lymphocyte infiltration in the lungs following B. pertussis infection of IL-4-defective mice but not in wild-type mice immunized with acellular vaccine. These data implicate that IL-4 is an important regulator of inflammatory cell requirement. In other animal studies, severe hypercholesterolemia was associated with the Th₂-oriented immune response, a reaction with increased IL-4 levels in the atherosclerotic lesions. In addition to the involvement of inflammation control, IL-4 acts on macrophages in vitro inducing macrophage M₂ (anti-inflammatory) alternative activation.

Interleukin-10 (IL-10) is the master anti-inflammatory cytokine, which is associated with disease severity in patients with infectious disease. IL-10 has potent deactivating properties in macrophages and T cells and modulates many cellular processes that may interfere with the development and stability of the atherosclerotic plaque.

CD206 is mannose receptor of a C-type lectin and present on the surface of macrophages and immature dendritic cells, that plays a role in both innate and adaptive immune systems. It is often used as a marker of M₂ (anti-inflammatory) macrophages. Innate and adaptive immune responses work together to restrain or eliminate hepatitis B virus in the liver (Dai K, Huang L, Sun X, et al. Hepatic CD206-positive macrophages express amphiregulin to promote the immunosuppressive activity of regulatory T cells in HBV infection. J Leuko Biol 98:1071-1080, 2015). They reported that CD206 macrophage plays an important role in modulating regulatory T-cell function and subsequently restrains the antiviral activity of CD8⁺ T cells. For example, CD206 has a role consistent with scavenging and degradation of ricin toxin, inhibiting cellular uptake and toxicity in vivo. Thus, strategies that optimize CD206-mediated uptake by macrophages in target tissues during infection are an attractive approach. Since adipose tissue resident macrophage have important role in the maintenance of tissue homeostasis and regulate insulin sensitivity by secreting pro-inflammatory or anti-inflammatory cytokines, Nawaz et al. (Nawaz A, Aminuddin A, Kado T, et al. CD206⁺ M2-like macrophages regulate systemic glucose metabolism by inhibiting proliferation of adipocyte progenitors. Nature communications 8.1 (2017): 286) demonstrated that CD206 M₂-like macrophages in adipose tissues create a microenvironment that inhibits growth and differentiation of adipocyte progenitors that control adiposity and systemic insulin sensitivity. CD206 also contributes to tumor immunosuppression, angiogenesis, metastasis, and relapse. Zhang et al. (Zhang C, Yu X, Gao L, et al. Noninvasive imaging of CD206-positive M2 macrophages as an early biomarker for post-chemotherapy tumor relapse and lymph mode metastasis. Theranostics 7 (17): 4276-4288, 2017) demonstrated that CD206⁺ M₂ macrophage-targeted imaging methods allowed for noninvasively prediction of post-chemotherapy tumor relapse and has the sensitivity to detect metastatic lymph nodes in vivo which helps with the rational design of cancer therapeutics. Others (Azad A K, Rajaram M V S, Metz W L, et al. Γ-Tilmanocept, a new radiopharmaceutical tracer for cancer sentinel lymph nodes, binds to the mannose receptor (CD206). J Immunol 195: Pages 1-11, 2015) also reported that identification of CD206 as the γ-tilmanocept-binding receptor enabled for designing receptor-targeted advance imaging agents and therapeutics for cancer and other diseases. Thus, CD206 is very useful biomarker and therapeutic agent for cancer diagnosis and therapeutic analysis. CD206⁺ M₂ macrophages also express heme oxygenase-1 (HO-1) that is required for prevention of diabetes-induced delayed gastric emptying (gastroparesis). Thus, induction of HO-1 in macrophages might be a therapeutic option for patients with diabetic gastroparesis as well as prevention of atherosclerosis.

CD11c is a cell-surface integrin expressed on a subset of circulating T-cells activated during bacterial infection and has been used as a marker of M₁ macrophages. CD11c levels are increased in adipose tissue and blood in mice and humans and contributes to insulin resistance associated obesity including inflammation. CD11c also plays an important role in monocyte recruitment and atherosclerosis development in a mouse model of hypercholesterolemia.

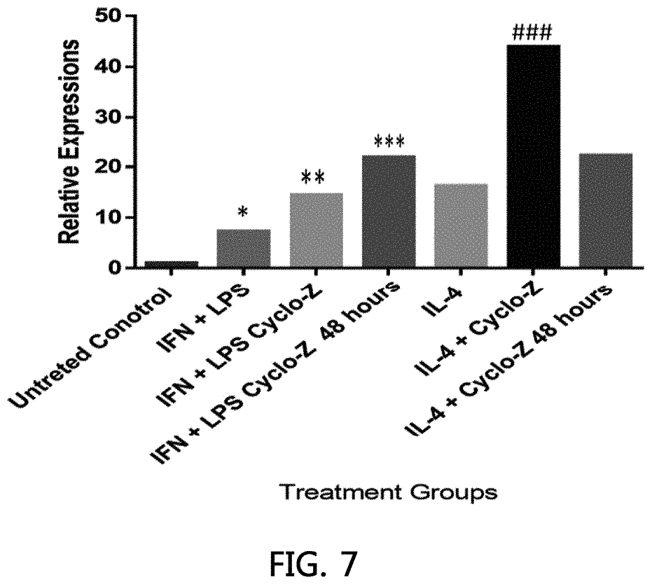

Based on these facts, the present inventors determined the effects of treatment with LPS/IFN (M₁) and IL-4 (M₂) in combination with Cyclo-Z on expression of CD11c (M₁ marker) and CD206 (M₂ marker) in RAW264.7 macrophage cells, TNFα in both macrophages and VSMC cultures, and IL-10 expression in VSMCs. As shown in FIG. 7, IFN/LPS M₁ treatment showed negative effects on CD206 expression as expected since CD206 is a M₂ macrophage marker. FIG. 7 shows that M₁ (IFN/LPS) treatment increased CD11c expression (marker of M₁ macrophage) and Cyclo-Z treatment further increased CD11c expressions in a time-dependent manner. Under inflammatory M₁ conditions (LPS/IFN), CD11c (in macrophages) and IL-10 (in VSMCs) levels increased compared to controls; however, TNFα levels increased in VSMCs, but decreased in macrophages with Cyclo-Z treatment (FIGS. 4-7). Cyclo-Z treatment further increased CD11c levels in a time-dependent manner in macrophages (FIG. 7). Cyclo-Z addition reduced IL-10 levels under M₁ conditions in VSMCs (FIG. 4). Since IL-10 is the master anti-inflammatory cytokines, Cyclo-Z might have the ability to nullify inflammatory activity of LPS/IFN and IL-10 is no longer needed to stop inflammation when Cyclo-Z is present.

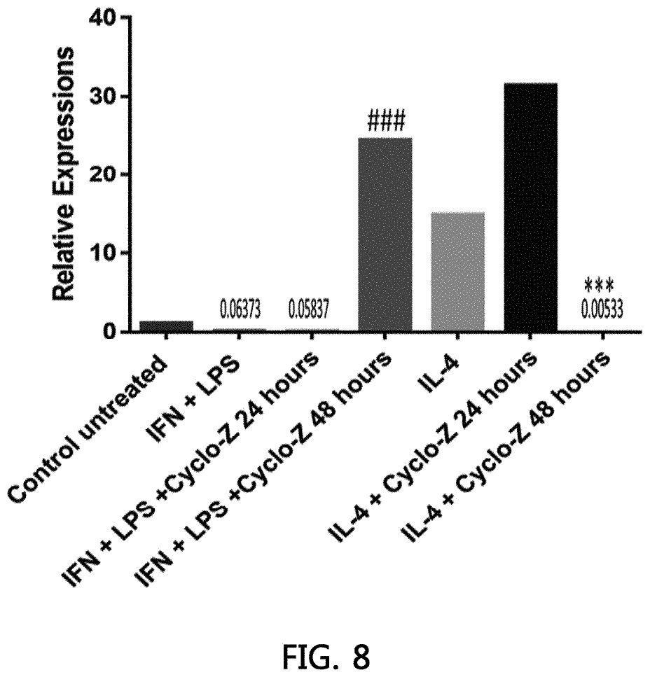

The key role of CD206 (mannose receptor) is to regulate the levels of molecules released into circulation during the inflammatory response and is used as a marker of M₂ (anti-inflammatory) macrophages. CD206 and TNFα are not elevated in the presence of IFN/LPS (M₁) treatment with and without CHP treatment at 24-hour treatment (FIGS. 6 and 8). However, at 48-hour incubation with Cyclo-Z, the CD206 levels significantly increased. Thus, Cyclo-Z treatment overcame the negative effect of inflammatory conditions converting the macrophages from an M₁ (inflammatory) to an M₂ (anti-inflammatory) phenotype (FIG. 7) and would be expected to improve the innate and adaptive immune system.

IL-4 plays an important role in the development of certain immune disorders, particularly allergies and some autoimmune diseases. Immune responses and inflammation play certain roles in type 2 diabetes development and complications. IL-4, along with other Th₂ cytokines, is involved in the airway inflammation observed in the lungs of patients with allergic asthma. IL-4 is also used to alternately activate macrophages to an M₂ (anti-inflammatory) phenotype which has a role in wound healing . IL-4 treatment induced CD206 expression as expected for M₂ macrophages and CycloZ treatment further increased CD206. Surprisingly, IL-4 addition in macrophage cultures increased expression of both M₁ and M₂ markers, CD11c (M₁), CD206 (M₂), and TNFα (M₁) (FIGS. 6-8). Addition of Cyclo-Z to IL-4 medium, increased levels of CD11c and CD206 but decreased TNFα levels decreased at 24 hrs. However, at 48-hour incubation with IL-4, CD11c was still increased but CD206 and TNFα levels decreased compared to immediate incubation results. These data (FIGS. 6-8) suggest that Cyclo-Z treatment modulates macrophage M₁/M₂ phenotype and cytokine metabolism depending on cell type, inflammatory state, and cellular biomedical conditions such as, diabetes, obesity, atherosclerosis, etc.

7. CD 206 expression in RAW267.4 macrophage cells treated with different combination of IFN, LPS, IL-4, and Cyclo-Z: Since CD206⁺ macrophages are important for wound healing in several diseases, additional experiments for the effects of LPS, IFN, IL-4, and Cyclo-Z on the CD206 expression were determined. As shown in FIG. 8, M₁ inflammatory conditions (IFN/LPS) without Cyclo-Z inhibit expression of CD206 M₂ (anti-inflammatory) marker and addition of Cyclo-Z did not show any effect on CD206 expression when incubated for 24 hours. However, after 48 hours incubation with Cyclo-Z under M₁ inflammatory conditions (IFN/LPS) showed significantly increased CD206 expression. In contrast, IL-4 induced CD206 expression, as expected for the M₂ macrophage phenotype, and increased even more with Cyclo-Z treatment at 24 hrs. However, with further incubation CD206 expression significantly decreased at 48 hrs. Thus, Cyclo-Z is an anti-inflammation agent inducing M₂ anti-inflammatory macrophages, and since CD206⁺ macrophages play a role in both innate and adaptive immune systems, stimulation of CD206⁺ macrophages by Cyclo-Z may ultimately be beneficial for a variety of diseases.

8. TNFα expression in macrophage cells treated with different combination of IFN, LPS, IL-4, and Cyclo-Z: M₁ treatment (IFN/LPS) reduced TNFα expression in macrophage cells. Addition of Cyclo-Z initially increased TNFα expression at 24 hr over control levels; but thereafter expression was reduced back to control levels at 48 hr (FIG. 6). However, M₂ (IL-4) treatment very significantly increased TNFα expression. Cyclo-Z treatment with IL-4 significantly reduced TNFα expression at 24 and further at 48 hrs. These data demonstrate that Cyclo-Z treatment inhibits cellular inflammation; although, M₂ (IL-4) conditions stimulated TNFα expression, Cyclo-Z treatment very significantly reduced TNFα mRNA levels. In addition, whereas, treatment for M₂ macrophage phenotype (IL-4) increased macrophage cell viability over M₀ (non-activated) cells, addition of Cyclo-Z with IL-4 further increase cell viability (Table 2). This increased viability effect may be more due to CHP as treatment with IL-4 and zinc alone (without CHP) decreased cell viability compared to IL-4 treatment alone.

9. Cell Viability in VSMCs and B-cell Activation in Spleen Cells: VSMCs cultured in the DMEM medium without serum for 7 days have reduced cell viability and cells numbers; however, survival of VSMCs is significantly increased when incubated with Cyclo-Z (100 μM CHP/10 μM Zn) compared to DMEM (no serum) control (FIG. 9). These data indicated that Cyclo-Z treatment prevents VSMC apoptosis, which is a prerequisite for many diseases including vascular calcification. The present inventors have also determined the effect of Cyclo-Z on LPS-induced B-cell activation in mouse spleen cells. Both LPS and Cyclo-Z (50 μM CHP/10 μM Zn) alone induced some B-cell activation/proliferation as indicated by nodules of dark cells in the culture (FIG. 10). However, when Cyclo-Z and LPS were added together, B-cell activation was much stronger. These data indicated that Cyclo-Z in combination with LPS enhances B-cell activation which is involved in immune response. Thus, Cyclo-Z is involved in enhancing immune system.

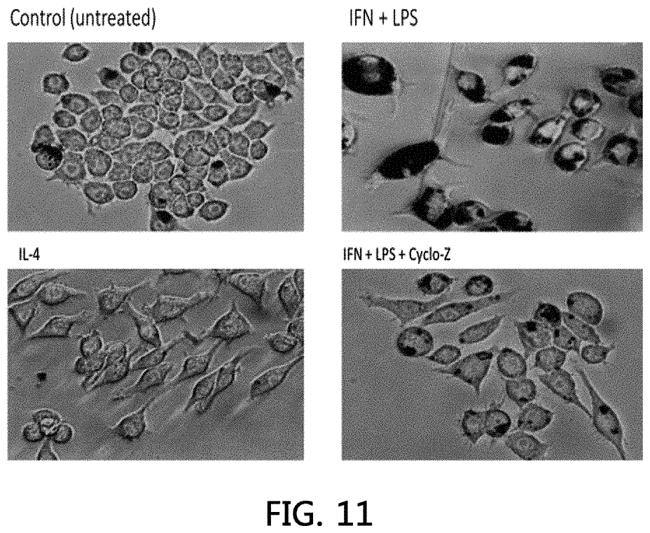

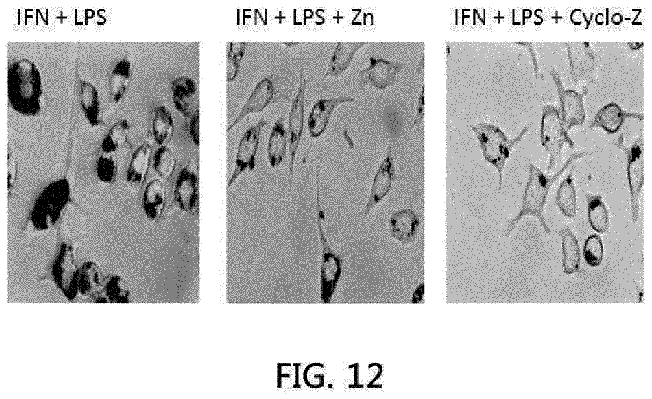

10. Cyclo-Z Induce M₂ Macrophage Activation: RT-PCR analysis of CD11c (M₁ marker) and CD206 (M₂ marker) mRNA expression levels indicates that Cyclo-Z induces M₂ (anti-inflammatory) activation of macrophages (FIGS. 7-8). The present inventors have also looked at activity of the M₁ and M₂ macrophages in an in vitro charcoal phagocytosis assay. Inflammatory M₁ macrophages (IFN/LPS) will phagocytize charcoal dust and becoming darkly stained; whereas, anti-inflammatory M₂ macrophages (IL-4) do not pick up the charcoal and remain clear (FIG. 11). As a control, non-activated M₀ macrophages may occasionally pick up a little charcoal powder. Cyclo-Z added during activation of macrophages with (IFN/LPS) prevents the conversion of the M₀ macrophages to M₁ macrophages, and we see a reduced level of charcoal in cells more closely resembling M₀ macrophage levels of phagocytosis. To compare between CHP and Zinc effects in Cyclo-Z on charcoal binding activities, we determined the phagocytosis activity of CHP or Zinc alone during activation of M₁ macrophages. Zinc was somewhat effective at inhibiting charcoal uptake, but not as effective as the combination of CHP and Zinc in Cyclo-Z (FIG. 12).

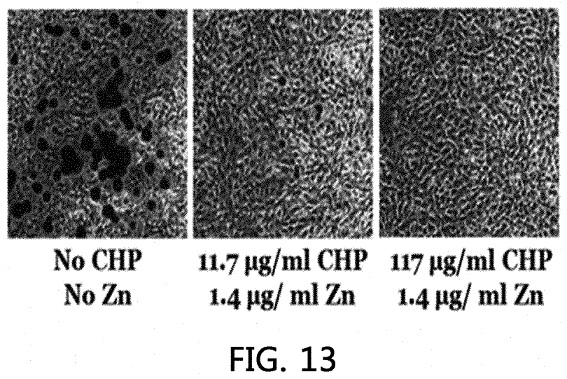

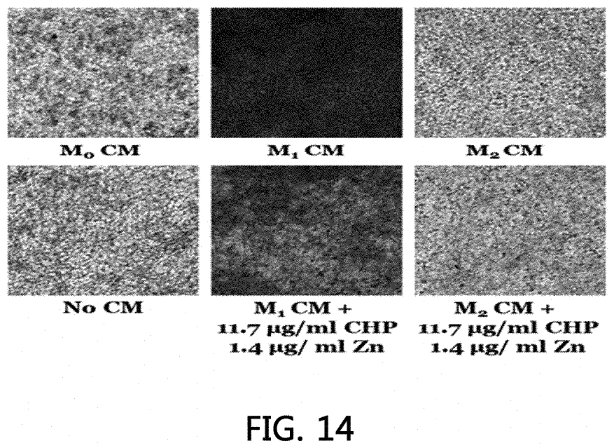

11. Inhibition of VSMC calcification with Cyclo-Z: Vascular calcification is associated with major adverse cardiovascular events and with a significant increase in all-cause mortality and atherosclerotic plaque rupture. Since mineralization of VSMCs with calcium is one of the main causes of atherosclerosis, the present inventors measured the effects of Cyclo-Z on calcification of VSMCs. In this in vitro ectopic calcification assay, VSMCs deposit mineral if calcium and phosphate concentrations are at least 2 mM each. Calcification is indicated by staining with Alizarin Red. Addition of Cyclo-Z in the medium will inhibit calcification over a 6-week period (FIG. 13). Furthermore, mineral is quickly deposited (1 week) if VSMCs are incubated with conditioned medium (CM) from cultured RAW264.7 macrophages activated to the pro-inflammatory M₁ phenotype (with IFN/LPS) in comparison to CM from RAW264.7 cells that are not activated (M₀) or alternative activated to anti-inflammatory M₂ phenotype (with IL-4). The ability of M₁CM to induce mineralization is reduced if Cyclo-Z is present during M₁ macrophage activation (FIG. 14). These data (FIGS. 13-14) indicated that Cyclo-Z treatment directly inhibits VSMC calcification and calcification activity in CM of M₁ macrophages. Thus, these data further indicate that Cyclo-Z treatment can prevent inflammation-induced VSMC calcification in vitro and suggests that Cyclo-Z intake in humans can strongly inhibit calcification and be used for prevention and treatment of atherosclerosis.

Accordingly, the present invention provides a method of preventing or treating atherosclerosis in a subject, the method comprising administering a therapeutically effective amount of a composition including a zinc salt and cyclo-Hispro to the subject.

In another aspect, the present invention provides a composition for preventing or treating atherosclerosis, comprising a composition including a zinc salt and cyclo-Hispro.

In still another aspect, the present invention provides a method of inhibiting inflammatory M₁ macrophage and/or inducing anti-inflammatory M₂ macrophage activation, comprising administering a therapeutically effective amount of a composition including a zinc salt and cyclo-Hispro.

In yet another aspect, the present invention provides a composition for inhibiting inflammatory M₁ macrophage and/or inducing anti-inflammatory M₂ macrophage activation, comprising a zinc salt and cyclo-Hispro.

In still another aspect, the present invention provides a method for preventing or treating of other metabolic diseases, neurodegenerative diseases or obesity in a subject, comprising administering a therapeutically effective amount of a composition including a zinc salt and cyclo-Hispro to the subject.

In yet another aspect, the present invention provides a composition for preventing or treating of other metabolic diseases, neurodegenerative diseases, or obesity in a subject, comprising a zinc salt and cyclo-Hispro.

In the method or the composition according to the present invention, the zinc salt may comprise a zinc cation and an anion.

In the method or the composition according to the present invention, the weight ratio of the zinc salt to cyclo-Hispro may be 1:0.1 to 10.

Alternatively, in the method or the composition according to the present invention, the molar concentration ratio of the zinc salt to cyclo-Hispro may be 1:1 to 1:15, preferably 1:2 to 1:13, more preferably 1:5 to 1:10.

In the method of the composition according to the present invention, the other metabolic diseases may be selected from the group consisting of rheumatoid arthritis, asthma, diabetes mellitus, cancer, macular degeneration, immune system diseases, inflammation, osteoporosis, and inflammatory bowel diseases, and the neurodegenerative disease may be Alzheimer's disease.

In the method or the composition according to the present invention, each ingredients of the composition can be included in purified form. By the use of the term “purified”, it is intended to mean that the ingredients of the composition are in a form enriched relative to the form in which they can be obtained from nature, such as in a prostate extract. The purified ingredients can be obtained either by enriching from a natural source thereof, or by a chemically synthetic method. Thus, the use of the term “purified” does not necessarily imply that these ingredients are completely free, or even substantially free, of other components. Nevertheless, a “purified” ingredient is enriched relative to its concentration in a natural prostate extract.

As used herein, the term “Cyclo-Z” means a composition comprising a zinc salt and cyclo-Hispro(CHP). CHP refers to a naturally occurring circular dipeptide consisting of histidine-proline, a metabolite of thyrotropin-releasing hormone (TRH), or to a physiologically active dipeptide that may be obtained from TRH metabolism or synthesized de novo in the body, and is a substance widely distributed in the brain, spinal cord, gastrointestinal tract, and the like.

In the method or the composition according to the present invention, the zinc being used may in the form of a pharmaceutically acceptable salt.

The pharmaceutically acceptable salt may be an acid addition salt formed by a free acid. The acid addition salt is prepared by a conventional method such as of dissolving a compound in an excess of an aqueous acid solution and precipitating the salt using a water-miscible organic solvent such as methanol, ethanol, acetone, or acetonitrile. Equimolar amounts of the compound and the acid or alcohol (e.g., glycol monomethyl ether) dissolved in water may be heated, followed by evaporating the mixture to dryness; or the precipitated salt may be acquired through suction filtration.

The free acid being used in this case may be either an inorganic acid or an organic acid. Examples of usable inorganic acid include hydrochloric acid, phosphoric acid, sulfuric acid, nitric acid, and tartaric acid, and examples of usable organic acid include methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, and hydroiodic acid, but the present invention is not limited thereto.

In addition, a pharmaceutically acceptable metal salt may be prepared using a base. An alkali metal salt or alkaline earth metal salt is obtained by, for example, dissolving zinc in an excess amount of an alkali metal or alkaline earth metal hydroxide solution, filtering the non-soluble compound salt, and evaporating and drying the filtrate. Although it is particularly preferable from a pharmaceutical point of view that the metal salt being produced in this case be a sodium salt, potassium salt, or calcium salt, the present invention is not limited thereto. In addition, a corresponding silver salt may be obtained by reacting the alkali metal salt or alkaline earth metal salt with a suitable silver salt (e.g., silver nitrate).

The pharmaceutical composition prepared according to the present invention preferably can be packaged in tablet or capsule form by procedures that are well known in the pharmaceutical arts. As referred to herein, numerical values for zinc represent masses or concentrations of the zinc component of a zinc salt. Examples of zinc salts useful in connection with the invention include zinc chloride and zinc sulfate.

In the method or the composition according to the present invention, the composition may further comprise an appropriate carrier, excipient, or diluent according to a conventional method. Examples of carriers, excipients and diluents that may be contained in the composition of the present invention include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, and mineral oil.

The composition according to the present invention may be formulated in the form of oral preparations (e.g., powders, granules, tablets, capsules, suspensions, emulsions, syrups, aerosols), external preparations, suppositories, or sterile injection solutions by a conventional method and used.

Specifically, it may be prepared using generally used diluents or excipients such as fillers, extenders, binders, humectants, disintegrants, and surfactants. Solid formulations for oral administration include tablets, pills, powders, granules, capsules, and the like, which may be prepared by mixing at least one excipient, such as starch, calcium carbonate, sucrose, lactose, or gelatin, with the aforementioned compound. In addition to simple excipients, lubricants such as magnesium stearate and talc may also be used. Examples of liquid preparations for oral administration include suspensions, internally applied liquids, emulsions, and syrups, wherein the liquid preparations may include various excipients such as wetting agents, sweeteners, fragrances, and preservatives in addition to water and liquid paraffin, which are conventional simple diluents. Examples of formulations for non-oral administration include sterile aqueous solutions, non-aqueous solvents, suspensions, emulsions, freeze-dried preparations, and suppositories. Examples of the non-aqueous solvents and suspensions include propylene glycol, polyethylene glycol, vegetable oils such as olive oil, and injectable esters such as ethyl oleate. Examples of a suppository base include Witepsol, Macrogol, Tween 61, cacao butter, laurin butter, and glycerogelatin.

The total effective amount of the composition of the present invention may be administered to a subject in a single dose or by a fractionated treatment protocol of administering in multiple doses over a prolonged period of time. The composition of the present invention may vary in the content of active ingredient depending on the severity of the disease. In the method or the composition according to the present invention, the preferable dosage of the composition varies depending on the condition and body weight of the patient, the severity of the disease, the drug form, the administration route, and the period, but a person skilled in the art will be able to appropriately select the desired dosage of the composition.

The method or the composition of the present invention may be used alone or in combination with surgery, radiation therapy, hormone therapy, chemotherapy, or methods using biological response modifiers.

In the method or the composition according to the present invention, when the composition is formulated into tablets or capsules, each tablet or capsule may contain 2.0 mg to 100 mg of cyclo-Hispro. In addition, each of the tablets or capsules may contain from 10 mg to 100 mg of zinc.

The pharmaceutical dosage form of the composition being used in the present invention may be in the form of pharmaceutically acceptable salts of constituents of the composition, which may be used independently or as a suitable set thereof, or in combination with other pharmaceutically active compounds.

The composition of the present invention may be administered to mammals such as humans, non-human primates (e.g., monkeys, chimpanzees, orangutans), pets (e.g., dogs, cats), and farm animals (e.g., horses, cows, mice, rats, rabbits, guinea pigs) by various routes. Any mode of administration may be contemplated; for example, the composition may be administered through an oral, rectal, intravenous, intramuscular, subcutaneous, or intrauterine route, or by intracerebroventricular injection.

The term “administration” or “administering” used herein means to provide a predetermined substance to a patient in any appropriate manner, and the administration may be performed non-orally (e.g., the formulation is injected intravenously, subcutaneously, intraperitoneally, or topically) or orally. The dosage may vary depending on the patient's body weight, age, sex, health condition, diet, administration time, administration method, excretion rate, the severity of the disease, and the like. Examples of liquid preparations for oral administration of the composition of the present invention include suspensions, internally applied liquids, emulsions, and syrups, wherein the liquid preparations may contain various excipients such as wetting agents, sweeteners, fragrances, and preservatives in addition to water and liquid paraffin, which are conventional simple diluents. Examples of formulations for non-oral administration include sterile aqueous solutions, non-aqueous solvents, suspensions, emulsions, freeze-dried preparations, and suppositories. The pharmaceutical composition of the present invention may be administered by any device capable of moving active substances to target cells. Preferred modes of administration and formulations are intravenous injection, subcutaneous injection, intradermal injection, intramuscular injection, drip injection, and the like. The injectable preparation may be prepared using an aqueous solvent (e.g., physiological saline solution, Ringer's solution) or a non-aqueous solvent such as a vegetable oil, a higher fatty acid ester (e.g., oleic acid), or an alcohol (e.g., ethanol, benzyl alcohol, propylene glycol, glycerin), and it may contain a pharmaceutical carrier such as a stabilizer for preventing spoilage (e.g., ascorbic acid, sodium bisulfite, sodium pyrosulfite, BHA, tocopherol, EDTA), an emulsifier, a buffer for pH control, and a preservative for inhibiting microbial growth (e.g., phenylmercury nitrate, thimerosal, benzalkonium chloride, phenol, cresol, benzyl alcohol).

In the method or the composition according to the present invention, the composition may include a food composition. The food composition includes all types of a functional food, a nutritional supplement, a health food, a food additive, feed, and the like, and fed to animals including humans or livestock. The types of the food composition may be prepared in various forms according to conventional methods known in the related art.

The types of the food composition may be prepared in various forms according to conventional methods known in the related art. Common foods may be prepared by adding the Cyclo-Z to beverages (including alcoholic beverages), fruits and processed foods thereof (e.g., canned fruits, bottled fruits, jam, marmalade, and the like), fish, meat and processed foods thereof (e.g., ham, sausage, corn beef, and the like), bread and noodles (e.g., udon, buckwheat noodles, ramen, spaghetti, macaroni, and the like), fruit juices, various drinks, cookies, taffy, dairy products (e.g., butter, cheese, and the like), edible vegetative oils, margarine, vegetable proteins, retort foods, frozen foods, various condiments (e.g., soybean paste, soy sauce, sauces, and the like), and the like, but the present invention is not limited thereto. Also, the nutritional supplement may be prepared by adding the Cyclo-Z to capsules, tablets, pills, and the like, but the present invention is not limited thereto. In addition, the health functional food may, for example, be prepared into liquids, granules, capsules and powders so that the Cyclo-Z itself can be prepared in the form of teas, juices and drinks so as to ingest health drinks, but the present invention is not limited thereto.

To use the Cyclo-Z in the form of a food additive, the Cyclo-Z may also be prepared in the form of a powder or a concentrate, and used. Further, the Cyclo-Z may be prepared in the form of a composition by being mixed with active ingredients known to have an effect of preventing or treating atherosclerosis.

When the composition according to the present invention is used as the health drink composition, the health drink composition may further contain additional components such as various flavoring agents or natural carbohydrates as in conventional drinks. The aforementioned natural carbohydrates may include monosaccharides such as glucose, fructose, and the like; disaccharides such as maltose, sucrose, and the like; polysaccharides such as dextrin, cyclodextrin, and the like; and sugar alcohols such as xylitol, sorbitol, erythritol, and the like. Natural sweetening agents such as thaumatin, a stevia extract, and the like; synthetic sweetening agents such as saccharin, aspartame, and the like may be used as the sweetening agent. A ratio of the natural carbohydrate is generally in a range of approximately 0.01 to 0.04 g, preferably approximately 0.02 to 0.03 g per 100 mL of the composition of the present invention.

The Cyclo-Z may be included as the active ingredient of the food composition for preventing or treating atherosclerosis, or for inhibiting M₁ macrophage and/or inducing anti-inflammatory M₂ macrophage.

In this case, an amount of the Cyclo-Z refers to an amount effective for achieving the effect of preventing or treating atherosclerosis, but the present invention is not particularly limited thereto. For example, the amount is preferably in a range of 0.01 to 100% by weight, based on the total weight of the composition. The food composition of the present invention may be prepared by mixing the Cyclo-Z with other active ingredients known to be effective in the composition for preventing or treating atherosclerosis, or for inhibiting M₁ macrophage and/or inducing anti-inflammatory M₂ macrophage.

In addition to the aforementioned components, the health food of the present invention may contain various nutrients, vitamins, electrolytes, a flavoring agent, a coloring agent, pectic acid, a pectate, alginic acid, an alginate, organic acids, a protective colloidal thickening agent, a pH control agent, a stabilizing agent, a preservative, glycerin, an alcohol, or a carbonating agent. In addition, the health food of the present invention may include pulp for preparing a natural fruit juice, a fruit juice drink or a vegetable drink. Such components may be used alone or in combination. The ratio of such additives is not important, but the additives are generally chosen in a range of 0.01 to 0.1 parts by weight, based on 100 parts by weight of the composition of the present invention.

In the method or the composition according to the present invention, the composition may induce ROS formation from hydrogen peroxide (H₂O₂); factor-erythroid 2-related factor (Nrf2); and/or antioxygen hemoxygenase-1 (HO-1).

In the method or the composition according to the present invention, the composition may prevent apoptosis or cell death.

In the method or the composition according to the present invention, the composition may prevent vascular smooth muscle cells (VSMC) calcification.

Cardiovascular diseases are the leading cause of morbidity and mortality in the world. Atherosclerotic plaques, consisting of lipid-laden macrophages and calcification, develop in the coronary arteries, aortic valve, aorta, and peripheral conduit arteries and are hallmark of cardiovascular disease. Trion and van der Learse (Trion A, van der Learse A. Vascular smooth muscle cells and calcification in atherosclerosis. Am Heat J 147(5):808-814, 2004) reported that vascular calcification is prominent feature of atherosclerosis, but the mechanisms underlying vascular calcification are still obscure. However, VSMCs are currently considered to be responsible for the formation of vascular calcification and apoptosis of VSMCs appeared to be a key factor in the process, while other factors including cell to cell interactions (macrophages and VSMCs), lipids, inflammation, cytokine metabolism (IFN, LPS, TNFα) and anti-oxidant transcription factors such as Nrf2 are contributing factors in inducing or preventing atherosclerosis. Thus, atherosclerosis, a disease previously viewed as inevitably progressive, can be treated to significantly alter arterial lesions and reduce their clinical consequences.

As shown in the above, the present inventors have addressed several of these pro-atherogenic factors including oxidative stress, inflammation, and ectopic vascular calcification. The oxidation of low density lipoprotein (LDL) indicates the first step of atherosclerosis in cardiovascular diseases. The present invention have shown that Cyclo-Z can act as a direct (FIG. 1) and indirect anti-oxidant stimulating expression of Heme oxygenase-1 (FIG. 2) and activation of the Nrf2 anti-oxidant pathway (FIG. 3). Inflammation has a crucial role in pathogenesis of atherosclerosis which is accompanied by excessive fibrosis of the intima, fatty plaques formation, proliferation of smooth muscle cells, and migration of a group of cells such as monocytes, T cells, and platelets which are formed in response to inflammation. The present inventors have proven that Cyclo-Z treatment reduces expression of inflammatory cytokines and markers (FIGS. 4-8) and that Cyclo-Z treatment reduce plaque forming calcification in VSMCs (FIGS. 13-14).

Because atherosclerosis is due to chronic vascular inflammation, arterial plaque formation is best conceptualized as a convergence of bone biology with vascular inflammatory pathobiology. The clinical relevance of intima calcification is thought to be different from that of media calcification. Intima calcifications contribute to plaque vulnerability while media calcification contribute to vascular stiffness. For the clinical presentation, treatment and prognosis, inhibition of common patho-mechanisms between vascular calcification and atherosclerosis, such as chronic inflammation, may be needed to successfully treat both intimal and medial calcification. In general, soft tissue calcification arises in areas of chronic inflammation, possibly functioning as a barrier limiting the spread of the inflammatory stimuli. Multiple stimulators and inhibitors control progression of vascular calcification, and it is likely to involve inflammatory mediators emanating from cells located in the arterial wall or the surrounding adventitia. Matrix effects also control the progression of calcification. Future work will be needed to unravel the molecular events leading to calcification and the development of novel molecular-based therapies to prevent atherosclerosis are needed. Recently Espitia et al. (Espitia O, Chatelais M, Steenamn M, et al. Implication of molecular vascular smooth muscle cell heterogeneity among arterial beds in arterial calcification. PLOS One 13(1): e0191976: Pages 1-17, 2018) suggested that biological heterogeneity of resident vascular cells between arterial beds is critical in understanding plaque biology and calcification, which may have strong implications in vascular therapeutic approaches. The data shown in the present invention suggest that Cyclo-Z is involved in controlling many biomedical events that regulate calcification and atherosclerosis including the immune system, inflammation, calcification, anti-oxygenic effects, and modulation of cytokine metabolism as described above. Thus Cyclo-Z is the novel anti-atherosclerosis agent for the treatment of human cardiac diseases.

The invention provides kits including compositions of the invention, combination compositions and pharmaceutical formulations thereof, packaged into a suitable packaging material. A kit optionally includes a label or packaging insert including a description of the components or instructions for use of the components in vitro, in vivo, or ex vivo therein.

The term “packaging material” refers to a physical structure housing the components of the kit. The packaging material may be able to maintain the components sterilely, and it may be made of a material commonly used for such purposes (e.g., paper, corrugated fiber, glass, plastic, foil, ampules, vials, tubes, etc.)

Kits of the invention may include labels or inserts. Labels or inserts include “printed matter” (e.g., paper or cardboard), separate or affixed to a component, kit, or packing material (e.g., a box), or attached to an ampule, tube or vial containing a kit component. Labels or inserts may additionally include a computer-readable medium, such as a disk (e.g., floppy diskette, hard disk, ZIP disk), an optical disk such as CD- or DVD-ROM/RAM, DVD, MP3, magnetic tape, or electrical storage media such as RAM and ROM or hybrids of these such as magnetic/optical storage media, flash media or memory type cards.

Labels or inserts may include the identifying information of one or more components therein, dose amounts, and/or clinical pharmacology of the active ingredients including mechanism of action, pharmacokinetics and pharmacodynamics. Labels or inserts may include identifying information of the manufacturer, lot numbers, manufacturer location, and date.

Labels or inserts may include information on the condition, disorder, disease or symptom for which a kit component may be used. Labels or inserts may include instructions for the clinician or subject for using one or more of the kit components in accordance with a method, treatment protocol or therapeutic regimen. Instructions may include dosage amounts, frequency or duration, and instructions for carrying out any of the methods, treatment protocols or therapeutic regimes set forth herein. Exemplary instructions include instructions for treating atherosclerosis. Kits of the invention therefore may additionally include labels or instructions for carrying out any of the methods of the invention described herein including treatment methods.

Labels or inserts may include information on any benefit that a component may provide, such as a prophylactic or therapeutic benefit. Labels or inserts may include information on potential adverse side effects, such as warnings to the subject or clinician regarding situations where it would not be appropriate to use a particular composition. Adverse side effects could also occur when the subject has been, will be or is currently taking one or more other medications that may be incompatible with the composition, or the subject has been, will be or is currently undergoing another treatment protocol or therapeutic regimen which would be incompatible with the composition; therefore, instructions could include information regarding such incompatibilities.

Invention kits may additionally include other components. Each component of the kit may be enclosed within an individual container, and all the various containers may be within a single package. Invention kits may be designed for cold storage.

Hereinafter, the present invention will be described in more detail through examples. These examples are only for exemplifying the present invention, and it will be obvious to a person with ordinary skill in the art that the scope of the present invention is not to be interpreted as being limited by these examples.

### PREPARATION EXAMPLE

### Preparation of Cyclo-Z and CHP

The zinc salt used in the following examples was purchased from Sigma-Aldrich (St. Louis, Mo.), and the cyclo-Hispro used in the following examples was purchased from Bachem (Basel, Switzerland).

### Example 1

### Colorimetric Assay

In this colorimetric assay, DPPH functions as a free radical that can be inactivated by anti-oxidant compounds resulting in loss of color. CHP was tested at various concentrations by incubation with DPPH and exhibits concentration dependent anti-oxidant activity. Zinc was not tested alone since it was not completely soluble in the organic solvents needed for the assay. Ascorbic Acid functions as an antioxidant positive control.

As shown in FIG. 1, CHP in the Cyclo-Z formulation can act as a direct anti-oxidant in an in vitro anti-oxidant assay in a CHP concentration-dependent manner.

### Example 2

### 2.1. HO-1 mRNA and Nrf2 mRNA Expression in VSMCs

Heme oxygenase-1 (HO-1) and Nrf2 mRNA expression levels were determined in vascular smooth muscle cells (VSMCs) (ATCC® CRL-2797) by real time RT-PCR (rtRT-PCR) after 72 hr treatment with inflammatory M1 macrophage conditioned medium (M1CM) with or without Cyclo-Z (CZ; 50 or 100 μM/CHP/10 μM zinc).

As shown in FIGS. 2 and 3, when Cyclo-Z was added with BGP to VSMC cultures, relative quantity of HO-1 and Nrf2 slightly increased compared to the cells incubated with BGP alone. VSMC incubated in conditional medium collected from 48 hours old RAW264.7 cells culture with M₁ macrophage phenotype (inflammatory) (M₁CM) had decreased levels of HO-1 and Nrf2 mRNA. Addition of Cyclo-Z during generation of the M₁CM conditioned medium restored VSMC basal levels of Nrf2 expression, and significantly increased levels of HO-1 compared to levels of M₁CM medium alone.

### 2-2. Reactive Oxygen Species (ROS) Generation and Cell Viability in VSMCs

VSMC were seeded (1.25×10⁵ cells/well in 12-well plates) and incubated at 37° C. for 24 hr. Cultures were then subjected to various treatments (as shown in Table 1) to induce ROS for 45 minutes (at 37° C.) along with 10 μM CM-H2CFDA (Molecular Probes, Inc., Eugene, Oreg.). CM-H₂DCFDA is a chloromethyl derivative of H₂DCFDA, useful as an indicator for ROS in cells. Subsequent oxidation of CM-H2CFDA yields a fluorescent adduct that is trapped inside the cell. At the end of treatment period cells were trypsinized, washed, and analyzed by FACS for ROS (CM-H2CFDA fluorescence) and viability using a viability stain (BD Horizon Fixable Viability Stain 660; BD Biosciences, San Jose, Calif.).

The results are shown in Table 1.

TABLE 1
ROS Generation and Cell Viability in VSMCs Depending
on the Various Cytokine Treatments Analyzed by FACS

| | | ROS | Viability |
|---|---|---|---|
| Nos. | Treatments | Population | Population |
| 1 | Untreated | 0.06 | 80.88 |
| 2 | Untreated + CZ | 0.24 | 82.17 |
| 3 | LPS | 0.09 | 75.81 |
| 4 | LPS + CZ | 0 | 75.22 |
| 5 | HG (50 mM) | 0.04 | 61.63 |
| 6 | HG (50 mM) + CZ | 0.08 | 81.41 |
| 7 | HG (100 mM) | 0.04 | 63.9 |
| 8 | HG (100 mM) + CZ | 0.04 | 68.22 |
| 9 | H₂O₂ (1 μL) + CZ | 45.52 | 35.26 |
| 10 | H₂O₂ (1 μL) + CZ | 86.64 | 73.978 |
| 11 | H₂O₂ (5 μL) | 81.33 | 22.31 |
| 12 | H₂O₂ (10 μL) | 50.63 | 7.33 |

CZ: Cyclo-Z
HG: High Glucose

The Table 1 shows that Cyclo-Z treatment induces hydrogen peroxide to be metabolized to hydroxy radical and water and stimulate reactive oxygen species (ROS) production, and in VSMC cultures in which cells were treated with high glucose or hydrogen peroxide to simulate dysregulated glucose metabolism and oxidative stress, Cyclo-Z provide protection to the oxidative stress resulting in increased cell viability and numbers.

Although reactive oxygen species (ROS) are increased by the treatment with Cyclo-Z for hydrogen peroxide degradation (Table 1), Cyclo-(his-pro) (CHP) can act as a direct anti-oxidant in an in vitro anti-oxidant assay in a CHP concentration-dependent manner (FIG. 1). Thus, Cyclo-Z treatment may be a very important anti-oxidant agent in the treatment of various metabolic diseases thru acting as a potent pro-oxidant which generate reactive oxygen radicals via a transition metal (zinc) catalyzed reaction, or indirectly to modulate redox status by upregulating of anti-oxidant proteins and enzymes.

### 2-3. IL-10 and TNFα mRNA Expression in VSMCs

IL-10 and TNFα mRNA expression levels were determined in same RNA treatment groups and samples as indicated in FIG. 2 HO-1 mRNA expression.

As shown in FIGS. 4 and 5, when Cyclo-Z was added with BGP to VSMC cultures, relative quantity of IL-10 levels very significantly decreased (FIG. 4), however, TNFα levels did not change compared to the cells incubated with BGP alone (FIG. 5).

When M₁ macrophage conditioned medium was incubated with VSMCs, IL-10 and TNFα levels increased respectively, however, with addition of Cyclo-Z IL-10 and TNFα mRNA levels significantly decreased (FIGS. 4 and 5).

### Example 3

### 3-1. Effects of Inflammatory (M₁) or Anti-Inflammatory (M₂) Activation on Levels of TNFα, CD11c and CD206 mRNA Expression in the RAW264.7 Macrophase Cell Line

TNFα, CD11c and CD206 mRNA levels were determined by real time rtRT-PCR in cultures of RAW264.7 macrophage cells (ATCC® TIB-71) activated to either inflammatory M1 macrophage phenotype (with LPS/IFN) or to the anti-inflammatory M2 phenotype (with IL-4) in the presence or absence of Cyclo-Z treatment for 24 or 48 hrs. CD11c and CD206 are used as a marker of inflammatory M₁ macrophage activation and inflammatory M₂ macrophage activation respectively.

As shown in FIG. 6, M1 treatment (IFN+LPS) did not significantly induce TNFα mRNA; however, M2 treatment (IL-4) did induce TNFα mRNA. Macrophage expression of TNFα mRNA was inhibited by Cyclo-Z at both 24 and 48 hr under anti-inflammatory M2 conditions.

FIG. 7 shows that M₁ treatment increased CD11c expression (marker of M₁ macrophage) and Cyclo-Z treatment further increased CD11c expressions in a time-dependent manner. Under inflammatory M₁ conditions, CD11c (in macrophages) levels increased compared to controls. IL-4 addition in macrophage cultures increased expression of CD11c. Addition of Cyclo-Z to IL-4 medium, increased levels of CD11c at 24 hrs. At 48-hour incubation with IL-4, CD11c was still increased compared to immediate incubation results.

FIG. 8 shows that CD206 are not elevated in M₁ treatment with and without Cyclo-Z treatment at 24-hour treatment, however, at 48-hour incubation with Cyclo-Z, the CD206 levels significantly increased. IL-4 addition in macrophage cultures increased expression of CD206. Addition of Cyclo-Z to IL-4 medium, increased levels of CD206 at 24 hrs. However, at 48-hour incubation with IL-4, CD206 levels decreased compared to immediate incubation results.

These data (FIGS. 6-8) suggest that Cyclo-Z treatment modulates macrophage M₁/M₂ phenotype and cytokine metabolism depending on cell type, inflammatory state, and cellular biomedical conditions such as, diabetes, obesity, atherosclerosis, etc.

### 3-2. FACS Analysis of Cell Viability in Cultures of M₂ Activated RAW246.7 With Cyclo-Z or Cyclo-Z Components (CHP or Zinc)

RAW264.7 macrophage cells, untreated and treated with IL-4 (20 ng/ml), were stained with 1:1000 diluted viability stain (BD Horizon Fixable Viability Stain 660). Cells were analyzed by FACS and gated for unstained (viable) and stained (non-viable) populations.

The results are shown in Table 2.

TABLE 2
Cell Viability in cultures of M₂ Activated
RAW246.7 with Cyclo-Z or Cyclo-Z
Components (CHP or Zinc)

| | Percent | Percent |
|---|---|---|
| TREATMENT | Viability (Stained) | Non-Viability |
| Untreated Control | 37.95 | 4.88 |
| IL-4 | 68.97 | 17.63 |
| IL-4 + CHP | 73.63 | 14.13 |
| IL-4 + Zn | 58.47 | 33.49 |
| IL-4 + CHP + Zn | 75.37 | 16.44 |

The Table 2 shows that the treatment for M2 macrophage phenotype (IL-4) increased macrophage cell viability over M0 (non-activated) cells, addition of Cyclo-Z with IL-4 further increase cell viability. This increased viability effect may be more due to CHP as treatment with IL-4 and zinc alone (without CHP) decreased cell viability compared to IL-4 treatment alone.

### Example 4

### Effect of Cyclo-Z on VSMCs Cell Viability in Serum-Free Cultures

VSMCs (ATCC® CRL-2797) were plated in medium with or without serum for 1 week to induce oxidative stress and cell death programs.

VSMC were seeded in T25 flasks (1×10⁶ cells) and incubated for 24 hr in regular growth medium (DMEM+10% FBS) at 37° C. After 24 hr, medium was aspirated off, and replaced with DMEM alone (no FBS) supplemented with or without Cyclo-Z as indicated. Medium (DMEM no FBS) was changed once (on day 3) during incubation period. Cells were observed for 7 days and photographed.

As shown in FIG. 9, cells from cultures with Cyclo-Z (right in FIG. 9) had increased cell viability and numbers in comparison to those without Cyclo-Z (left in FIG. 9).

### Example 5

### Effect of Cyclo-Z on B-Cell Activation in Mouse Spleen Tissues

Mouse spleen cells were isolated from wild type C57BL/6J mice (The Jackson Laboratory; strain 000632) and plated under standard cell culture conditions in the presence or absence of Cyclo-Z (50 μM CHP/10 μM zinc) and/or LPS for 4 days. Nodules indicating increased B-cell activation and proliferation were visualized by microscopy.

As shown in FIG. 10, both LPS (1 μg/ml) and Cyclo-Z (50 μM CHP/10 μM Zn) alone induced some B-cell activation/proliferation as indicated by nodules of dark cells in the culture. However, when Cyclo-Z and LPS were added together, B-cell activation was much stronger.

### Example 6

### 6-1. Effects of Cyclo-Z on Phagocytic Activity of Inflammatory (M₁) or Anti-Inflammatory (M₂) Activated RAW264.7 Macrophage Cells

RAW264.7 macrophage cells (ATCC® TIB-71) were activated to either inflammatory M₁ macrophage phenotype (with 1 μg/ml LPS/20 ng/ml IFN) or to the anti-inflammatory M₂ phenotype (with 20 ng/ml IL-4) in the presence or absence of Cyclo-Z (50 μM CHP/10 μM Zn) treatment. Charcoal dust was added to the culture as a marker of M₁ phagocytosis activity.

As shown in FIG. 11, M₁ macrophages become dark after charcoal uptake; whereas, anti-inflammatory M₂ macrophages do not exhibit phagocytosis activity and remain clear. As a control, non-activated M₀ macrophages may occasionally pick up a little charcoal powder. Cyclo-Z added during activation of macrophages with (IFN/LPS) prevents the conversion of the M₀ macrophages to M₁ macrophages, and a reduced level of charcoal in cells more closely resembling M₀ macrophage levels of phagocytosis.

### 6-2. Effects of CHP or Zinc Alone on Phagocytic Activity of Inflammatory (M₁) Activated RAW264.7 Macrophage Cells

To compare between CHP and Zinc effects in Cyclo-Z on charcoal binding activities, we determined the phagocytosis activity of CHP (50 μM CHP) or Zinc (10 μM Zn) alone or Cyclo-Z during activation of M₁ macrophages (with 1 μg/ml LPS/20 ng/ml IFN).

As shown in FIG. 12, Zinc alone was somewhat effective at inhibiting charcoal uptake in M₁ macrophages, but not as effective as the combination of CHP and Zinc in Cyclo-Z. CHP alone did not show any effects on charcoal uptake (data not shown).

### Example 7

### 7-1. Inhibition of VSMC Calcification With Cyclo-Z

VSMCs (ATCC® CRL-2797) calcification was directly induced with 2.5 mM CaCl₂ and 10 mM BGP in 6-week-old VSMC cultures directly treated with or without Cyclo-Z at indicated concentrations (FIG. 13). Mineral was visualized after Alizarin Red staining of calcium deposits.

As shown in FIG. 13, addition of Cyclo-Z in the medium inhibited calcification over a 6-week period.

### 7-2. Calcification of VSMCs Dependent on Inflammatory Factors Present in Conditioned Medium from M₁ (Inflammatory) or M₂ (Anti-inflammatory) RAW264.7 Cell Cultures With or Without Cyclo-Z Added During Macrophage Activation

VSMCs calcification (1 week) with 48 hr CM (1:1 mix in DMEM) from M₀ (not activated), M₁ (pro-inflammatory), or M₂ (anti-inflammatory) macrophages. Cyclo-Z was present in macrophage culture during activation as indicated (FIG. 14). Mineral visualized after Alizarin Red staining of calcium deposits.

As shown in FIG. 14, mineral is quickly deposited (1 week) if VSMCs are incubated with conditioned medium (CM) from cultured RAW264.7 macrophages activated to the pro-inflammatory M₁ phenotype (with IFN/LPS) in comparison to CM from RAW264.7 cells that are not activated (Mₒ) or alternative activated to anti-inflammatory M₂ phenotype (with IL-4). The ability of M₁CM to induce mineralization is reduced if Cyclo-Z is present during M₁ macrophage activation.

These data (FIGS. 13-14) indicated that Cyclo-Z treatment directly inhibits VSMC calcification and calcification activity in CM of M₁ macrophages. Thus, these data further indicate that Cyclo-Z treatment can prevent inflammation-induced VSMC calcification in vitro and suggests that Cyclo-Z intake in humans can strongly inhibit calcification and be used for prevention and treatment of atherosclerosis.

1. A method of preventing or treating atherosclerosis in a subject, the method comprising administering a therapeutically effective amount of a composition including a zinc salt and cyclo-Hispro to the subject.

2. The method of claim 1, wherein the zinc salt comprises a zinc cation and a zinc anion.

3. The method of claim 1, wherein the weight ratio of the zinc salt to the cyclo-Hispro is 1:0.1 to 10.

4. The method of claim 1, wherein the composition is in a form of a tablet or capsule.

5. The method of claim 4, wherein each tablet or capsule contains from 2 mg to 100 mg of cyclo-Hispro.

6. The method of claim 4, wherein each tablet or capsule contains from 10 mg to 100 mg of zinc.

7. The method of claim 1, wherein the composition induces a reactive oxygen species (ROS) formation from hydrogen peroxide (H₂O₂).

8. The method of claim 1, wherein the composition induces a production of a factor-erythroid 2-related factor (Nrf2).

9. The method of claim 1, wherein the composition induces a production of antioxygen hemoxygenase-1 (HO-1).

10. The method of claim 1, wherein the composition prevents an apoptosis or cell death.

11. The method of claim 1, wherein the composition prevents a vascular smooth muscle cells (VSMC) calcification.

12. A composition comprising a zinc salt and cyclo-Hispro.

13. A method of inhibiting inflammatory M₁ macrophage and/or inducing anti-inflammatory M₂ macrophage activation in a subject, comprising administering an effective amount of the composition of claim 12.

14. A method for preventing or treating of metabolic diseases, neurodegenerative diseases, or obesity in a subject, comprising administering an effective amount of the composition of claim 12.

15. The method of claim 14, wherein the metabolic diseases are selected from the group consisting of rheumatoid arthritis, asthma, diabetes mellitus, cancer, macular degeneration, immune system diseases, inflammation, osteoporosis, and inflammatory bowel diseases; and the neurodegenerative disease is Alzheimer's disease.

16. The method of claim 15, wherein the zinc salt comprises a zinc cation and an anion.

17. The method of claim 15, wherein the composition is in the form of a tablet or capsule.

18. The method of claim 17, wherein each tablet or capsule contains from 2 mg to 100 mg of cyclo-Hispro.

19. The method of claim 17, wherein each tablet or capsule contains from 10 mg to 100 mg of zinc.
